(19) United States
(12) Patent Application Publication
(10) Pub. No.: US 2004/0226061 A1
(43) Pub. Date: Nov. 11, 2004

(54) Goose creek tomato

(76) Inventor: Jimmy Williams, Los Angeles, CA (US)

Correspondence Address: MaryDilys S. Anderson, Ph.D., Wolf, Greenfield & Sacks, P.C., 600 Atlantic Avenue, Boston, MA, 02210, US

(21) Appl. No.: 10/431,792

(22) Filed: May 8, 2003

(51) Int. Cl. A01H 5/10; A01H 1/00; A01H 5/00; A01H 5/08; C12N 5/04

(52) U.S. Cl. 800/317.4; 800/298; 435/411; 435/423; 800/260

(57) ABSTRACT

The invention relates to a distinct tomato line, designated the Goose Creek tomato line. Additionally, the invention relates to seed and fruit of the Goose Creek tomato line as well as methods of producing the plant, fruit, and seed of the Goose Creek tomato line. The invention also relates to Goose Creek-derived tomato plants, fruit, and seed and methods of producing the Goose Creek-derived plants, fruit, and seed.

### FIELD OF THE INVENTION

The invention relates to a distinct tomato line with dual ripening stages, which is designated the Goose Creek tomato line. Additionally, the invention relates in part to seed and fruit of the Goose Creek tomato line as well as methods of producing the plant, fruit, and seed of the Goose Creek tomato line and Goose Creek-derived tomato plants, fruit, and seed.

### BACKGROUND OF THE INVENTION

Tomatoes, which have the scientific name Lycopersicon esculentum, are believed to have originated in western South America and from there have been taken to various regions of the World and cultivated as a food source. The cultivated tomato is known to have arrived in the United States from several different sources including Mexico, Europe, and in some instances from Africa. Today tomatoes are grown around the World in many different environments. In more temperate regions, the tomato is grown as a perennial and in other regions with colder climates, tomatoes are grown as annuals.

Tomatoes are an important commercial crop in the United States, but also have great popularity with individual gardeners. Many varieties of tomatoes are available and there is a wide range of genetic variability between the different tomato types. In the United States and around the World today, tomato lines are available that possess combinations of many different characteristics including such features as color of flowers and fruit, size and taste of fruit, length of growth season, and leaf size and shape. Some characteristics such as firmness of ripe fruit, shelf-life, amenability to shipping and storage, and resistance to pests are very important considerations for commercially grown tomato lines. Additional characteristics such as size of fruit, color, and taste of fruit are important qualities sought by individual gardeners for smaller-scale production.

The currently available variety of tomato lines have been created through the use of plant breeding strategies and standard methods such as those cited in U.S. Pat. No. 6,414,226, the contents of which is incorporated herein by reference in its entirety.

The method of breeding for tomato lines depends on breeding characteristics of the lines (e.g. open pollination or hybrids). One method for identifying a desirable line is to observe the characteristics of the line relative to other experimental plants or standard cultivated lines. Observations can be made over several growing seasons to determine whether a given characteristic is stable and maintained in the line. Testing of a tomato line can provide information regarding its differences from, and superiority to other tomato cultivars.

### SUMMARY OF THE INVENTION

The Goose Creek tomato line has been developed from seed that was originally planted in the Gullah region of South Carolina. The original Gullah tomato plantings are believed to have been established with seeds brought into the region by African people who had been forcibly taken from Africa to the Gullah region of South Carolina. The seeds used for the original plantings from which the Goose Creek tomato line has been developed were closely held in a single family and passed down through that family for generations. Over the past 51 years seeds from the family legacy have been cultivated and bred to develop a consistent, reproducible line, which is referred to herein as the Goose Creek tomato line.

According to one aspect of the invention, tomato seed designated Goose Creek, wherein a sample of the seed has been deposited under American Type Culture Collection Accession No. ______, are provided. In some embodiments, tomato plants, or parts thereof, produced by growing the seed of the tomato plant are provided. Tomato plant parts include the pollen of the tomato plant, fruit from the tomato plant, ovules of the tomato plant, as well as leaves, stems, flowers, and roots of the tomato plant. In some embodiments, the tomato plants, or parts thereof, include physiological and morphological characteristics as described below.

In other embodiments, the invention includes a tissue culture of regenerable cells of the tomato plant. The cells can regenerate a plant capable of expressing all the morphological and physiological characteristics of the Goose Creek tomato plant. In some embodiments, the tissue culture includes cells or protoplasts from a tissue selected from the group consisting of leaves, pollen, embryos, roots, root tips, anthers, flowers, fruit, and seeds. In certain embodiments, a tomato plant regenerated from the tissue culture that is capable of expressing all the morphological and physiological characteristics of the Goose Creek tomato plant is provided.

In another aspect, the invention provides methods for producing a tomato seed. The methods include growing the Goose Creek tomato plant and harvesting the resultant seed. The invention also includes tomato seed produced according to these methods, as well as tomato plants, or parts thereof, produced from the seed. In some embodiments, the invention includes fruit of the tomato plant produced from the foregoing seeds.

According to another aspect of the invention, methods for producing Goose Creek-derived tomato seed are provided. The methods involve crossing a Goose Creek tomato plant with a non-Goose Creek tomato plant and harvesting the resultant tomato seed. In some embodiments of the invention, Goose Creek-derived tomato seed produced by the foregoing method is provided. Goose Creek-derived tomato plants, or parts thereof, produced by growing the foregoing tomato seed also are provided. In some embodiments, fruit of the foregoing tomato plant is provided.

Tissue cultures of regenerable cells of the Goose Creek-derived tomato plants also are provided. The cells can regenerate plants capable of expressing all the morphological and physiological characteristics of the Goose Creek-derived tomato plant. In certain embodiments, the tissue culture includes cells or protoplasts from a tissue selected from the group consisting of leaves, pollen, embryos, roots, root tips, anthers, flowers, fruit, and seeds.

The present invention also relates in part to tomato plants having within their pedigree the Goose Creek tomato line.

### DETAILED DESCRIPTION OF THE INVENTION

The invention relates in part to a tomato line, designated Goose Creek, that has a novel dual-ripening characteristic. The Goose Creek line described herein can also be used in combination with other tomato lines to produce novel tomato varieties. Another aspect of the invention includes the fruit and seed of the Goose Creek tomato line. As used herein, “seed” refers to one or more seeds. In addition, the invention relates to methods of using Goose Creek germplasm, protoplasm, and tissues to produce Goose Creek plants, fruits, seed, or parts thereof, and/or to produce any Goose Creek-derived plants, fruits, seed, or parts thereof. As used herein, the phrase “parts thereof,” means a subpart, for example, tissue of a fruit, or a tissue from a seed. This means that any part of a plant, fruit, or seed of the Goose Creek line, and any part of a plant, fruit, or seed of a line derived from the Goose Creek line is considered to be within the scope of the claimed invention. As used herein the term “derived from” means “arises from”, which includes either directly or indirectly. Thus, a plant derived from another plant may arise though standard plant breeding practices or may arise through standard tissue culture and plant regeneration methods, which are well known in the art.

The Goose Creek tomato line has been developed from seed obtained from the Gullah region of South Carolina. The Goose Creek tomato line was developed through plant breeding methods coupled with the selection and promotion of desired traits such as dual ripening, dual color stages, round fruit, good fruit firmness, early ripening, indeterminate growth habit, and other traits described in Table 1, which is adapted from the Protocol for Distinctness, Uniformity and Stability Tests, European Union, Community Plant Variety Office, CPVO-TP/44/2, Nov. 15, 2001, which is incorporated herein by reference in its entirety. The descriptions provided herein indicate various traits and terms associated with tomato plants, flowers, fruit, and seed. The definitions of characteristics (see Table 1) and terms are used to provided a clear understanding of the specification and are used in accordance with the standard terminology.

TABLE 1
Definitions and Delineations of Tomato Characteristics

| Characteristic | Type |
|---|---|
| Plant: Growth type | determinate or indeterminate |
| Leaf: attitude (middle third of plant) | semi-erect, horizontal, or |
| | semi-drooping |
| Leaf: length | short, medium, long |
| Keaf: width | narrow, medium, broad |
| Leaf: division of blade | pinnate or bipinnate |
| Leaf: size of leaflets in middle of leaf | very small, small, medium, |
| | large, or very large |
| Leaf: intensity of color | light, medium dark |
| Leaf: glossiness | weak, medium, strong |
| Leaf: blistering | weak, medium, or strong |
| Leaf: size of blisters | small medium or large |
| Leaf: attitude of petiole of leaflet in | semi-erect, horizontal, |
| relation to main axis | semi-relation |
| Inflorescence type | mainly uniparous, intermediate, |
| | mainly multiparous |
| Flower: pubescence of style | absent or present |
| Flower: color | yellow or orange |
| Peduncle: abscission layer | absent or present |
| Fruit: size | very small, small, medium, |
| | large, or very large |
| Fruit: ratio length/diameter | very small, small, medium, |
| | large, or very large |
| Fruit: shape in longitudinal section | flattened, slightly flattened, |
| | circular, rectangular cylindrical, |
| | elliptic, heart-shaped, obovate, |
| | ovate, or pear-shaped |
| Fruit: ribbing at stem end | weak, medium, strong, |
| | or very strong |
| Fruit: cross section | not round or round |
| Fruit: depression at peduncle end | absent or very weak, weak, |
| | medium strong, or very |
| | strong |
| Fruit: size of peduncle scar | very small, small, medium, |
| | large, or very large |
| Fruit: size of blossom scar | very small, small, medium, |
| | large, or very large |
| Fruit: shape at blossom end | indented, indented to flat, |
| | flat to pointed, or pointed |
| Fruit: size of core in cross section | very small, small, medium, |
| | large, or very large |
| Fruit: thickness of pericarp | thin, medium, or thick |
| Fruit: number of locules | only two, two or three, three |
| | or four, four, five or six, or |
| | more than six |
| Fruit: green shoulder (before maturity) | absent or present |
| Fruit: extent of green shoulder | small, medium, or large |
| Fruit: intensity of green color of | light, medium, or dark |
| shoulder | |
| Fruit: intensity of green color before | light, medium or dark |
| maturity | |
| Fruit: color at maturity | cream, yellow, orange, pink, |
| | red, or brownish |
| Fruit: color of flesh at maturity | cream, yellow, orange, pink, |
| | red, or brownish |
| Fruit: firmness | very soft, soft, medium, firm, |
| | or very firm |
| Time of flowering | early, medium, or late |
| Time of maturity | very early, early, medium, late, |
| | or very late |

Goose Creek Line Plant, Flower, and Fruit Characteristics

The following information on the characteristics of the Goose Creek tomato line was obtained through field observations. Parent line Goose Creek tomatoes (ATCC Accession No.: ______) were crossed and progeny seed was collected. Plants generated from the progeny seed were grown and produced plants, fruit, and seed of the Goose Creek tomato line. The characteristics described below were found to be present in the Goose Creek tomato line, which is uniform and stable for all traits. The Goose Creek tomato line comes true to seed.

The Goose Creek tomato line is an indeterminate growth type with long, medium-wide, semi-erect, bipinnate leaves. The leaves are medium green in color and have a medium level of glossiness. Leaf blistering is weak with small-sized blisters. The inflorescences are mainly multiparous with pubescence of style (hair) present. The flowers themselves are yellow in color and the plant exhibits early flowering. There is no abscission layer in the peduncle. The fruit vary in size, with both small and medium fruit present on the same plant. The ratio of length to diameter in the fruit is medium, and the shape of the fruit in longitudinal and cross section is round. There is weak ribbing of the fruit at the peduncle end and a medium depression present in the fruit at the peduncle end. The scarring on the fruit includes a medium peduncle scar and a small blossom scar. At the blossom end the fruit shape is flat.

The cross section of the core of the fruit is medium in thickness as compared to the total fruit diameter and the thickness of the pericarp is also medium. The fruit generally has 4, 5, or 6, locules. The fruit has a light green shoulder prior to maturity, and a light-green overall color at stages prior to maturity.

The fruit has an unusual characteristic of two distinct stages of ripeness with fruit at the first ripe stage having characteristic green shoulders and an orange coloration on the remainder of the fruit. At the second ripe stage the fruit have a characteristic overall red color. The fruit are edible at both ripe stages. The fruit in the first ripe stage exhibits a medium to firm texture with a citrus-like, fruity flavor. The fruit in the second ripe stage exhibits a soft to medium firmness and is higher in juice and Brix (sugar) content than is present in fruit at the first ripe stage. Thus, the fruit can be harvested at either of two different stages to yield the unique appearance, texture, and flavor properties characteristic of ripeness at each stage.

In addition to the Goose Creek tomato line described herein, the Goose Creek tomato line can also be crossed with other tomato lines (e.g., non-Goose Creek tomato lines) using standard breeding procedures to yield Goose Creek-derived tomato lines that include plants, fruit, and seed.

For example the invention includes methods for producing a Goose Creek-derived tomato plant. The methods include crossing Goose Creek tomato plant, a sample of seed of the line having been deposited under American Type Culture Collection accession number ______, with a second tomato plant to yield progeny tomato seed. The progeny tomato seed is then grown under plant growth conditions, to yield a Goose Creek-derived tomato plant. In addition, a Goose Creek-derived tomato plant can also be crossed with itself, with another Goose Creek tomato plant, or with another tomato plant (e.g. a non-Goose Creek tomato plant) to yield additional Goose Creek-derived progeny tomato seed. The seeds produced using these methods can then be grown to yield additional Goose Creek-derived tomato plants. These derivation procedures (i.e., the crossing and growing steps) can be optionally repeated to generate additional Goose Creek-derived tomato plants, fruit, and seed.

In addition to the use of standard plant breeding methods to produce Goose Creek-derived tomato plants, fruit, and seed, plant tissue culture methods can also be used to derive progeny of the Goose Creek-derived tomato plant and to produce Goose Creek-derived plants, fruit, and seed.

The above examples are provided to illustrate the invention but not to limit its scope. Other variants of the invention will be readily apparent to one of ordinary skill in the art and are encompassed by the appended claims. All publications and patents cited herein are hereby incorporated by reference in their entirety. All deposits made with the American Type Tissue Culture described herein are governed by the Budapest Treaty.

1. Tomato seed designated Goose Creek, wherein a sample of the seed has been deposited under American Type Culture Collection Accession No. ______.

2. A tomato plant, or part thereof, produced by growing the seed of claim 1.

3. Pollen of the tomato plant of claim 2.

4. Fruit from the tomato plant of claim 2.

5. An ovule of the tomato plant of claim 2.

6. A tomato plant, or part thereof, comprising the physiological and morphological characteristics of the tomato plant of claim 2.

7. A tissue culture of regenerable cells of the tomato plant of claim 2, wherein the tissue regenerates a plant capable of expressing all the morphological and physiological characteristics of the Goose Creek tomato plant.

8. A tissue culture according to claim 7, comprising cells or protoplasts from a tissue selected from the group consisting of leaves, pollen, embryos, roots, root tips, anthers, flowers, fruit, and seeds.

9. A tomato plant regenerated from the tissue culture of claim 7, capable of expressing all the morphological and physiological characteristics of the Goose Creek tomato plant.

10. A method for producing tomato seed comprising growing the tomato plant of claim 2 and harvesting the resultant seed.

11. Tomato seed produced according to claim 10.

12. A tomato plant, or part thereof, produced from the seed of claim 11.

13. Fruit of the tomato plant of claim 12.

14. A method for producing a Goose Creek-derived tomato seed comprising crossing the plant according to claim 2 with a non-Goose Creek tomato plant and harvesting the resultant tomato seed.

15. Goose Creek-derived tomato seed produced by the method of claim 14.

16. A Goose Creek-derived tomato plant, or part thereof, produced by growing the tomato seed of claim 15.

17. Fruit of the tomato plant of claim 16.

18. A tissue culture of regenerable cells of the tomato plant of claim 16, wherein the tissue regenerates a plant capable of expressing all the morphological and physiological characteristics of the Goose Creek-derived tomato plant.

19. A tissue culture according to claim 18, comprising cells or protoplasts from a tissue selected from the group consisting of leaves, pollen, embryos, roots, root tips, anthers, flowers, fruit, and seeds.
